(19) United States
(12) Patent Application Publication
(10) Pub. No.: US 2007/0003002 A1
(43) Pub. Date: Jan. 4, 2007

(54) Method for automatically setting and reconstructing the field of view along the inner boundaries of the thorax on a CT topogram

(76) Inventors: Ma Hao Chen, Shanghai (CN); Xiao Dong Xu, Shanghai (CN); Feng Zhao, Shanghai (CN)

Correspondence Address: HARNESS, DICKEY & PIERCE, P.L.C., P.O.BOX 8910, RESTON, VA, 20195, US

(21) Appl. No.: 11/476,833

(22) Filed: Jun. 29, 2006

(30) Foreign Application Priority Data
Jun. 30, 2005 (CN) 200510080217.9

(51) Int. Cl. H05G 1/60; A61B 6/00; G01N 23/00; G21K 1/12

(52) U.S. Cl. 378/4

(57) ABSTRACT

A method is disclosed for automatically setting and reconstructing the field of view along the inner boundary of the thorax on a CT topogram, the reconstructed field of view including exactly all the points on the inner boundaries of the thorax inside the scan area on the topogram. In an embodiment of the method, the search takes place from the body bones in the direction of the thorax, and the inner boundaries of the thorax of the patient are determined by comparing the CT values. An embodiment of method can be used for a precise, highly efficient and quick setting and reconstruction of the field of view.

### PRIORITY STATEMENT

The present application hereby claims priority under 35 U.S.C. §119 on Chinese patent application number CN 200510080217.9 filed Jun. 30, 2005, the entire contents of which is hereby incorporated herein by reference.

### FIELD

The present invention generally relates to a method for setting and reconstructing the field of view (termed “FOV” below for brevity) on the topogram (also termed “topo image”) for computer tomography (termed “CT” below for brevity). In particular, it may relate to a method for automatically setting the FOV along the inner boundaries of the thorax (contours) of a patient on a CT topogram.

### BACKGROUND

Before beginning to carry out the serial scanning or the spiral scanning, there is normally a need with CT units to create a positioning image for the patient in order to determine the scan area with the aid of this positioning image, and in order to be able to undertake the positioning of the image reconstruction. Subsequently, an x-radiation source is used to perform the scanning operation of the area of the patient's body to be examined so as to carry out the image reconstruction and to produce the medical image with the aid of the projection data.

During a topogram scan, the spherical tube, which serves as x-radiation source inside the CT unit, remains with its position unchanged, while the sickbed moves in order to obtain the topogram in this way. Reference may be made to FIG. 1 in this regard. In the relevant figure, the part designated by the number 110 is the area scanned in a typical topogram 100. The part designated by number 112 is the unscanned area in the topogram.

In the current prior art, the area to be scanned and the FOV are set on the topogram 100 designated above after the latter has been obtained, and this is illustrated by a rectangle 20. Under particular conditions, a parallelogram can also be used for the illustration. In this case, the scan area is determined along the side of the height of the patient's body 60 (that is to say the vertical side of the rectangle). The relevant examination area in the scan area and in the reconstructed image is acquired by using the scan area designated above, while the other areas of a patient's body are not scanned.

By contrast, the FOV is determined along the side of the width direction of the patient's body (that is to say the horizontal side of the rectangle). The FOV designated above is used to determine the display area of the image. Thus, the determination of position and size of the display of the area being examined on the image. The CT unit subsequently performs this serial scanning or the spiral scanning as well as the image reconstruction in accordance with the area designated above and set in a rectangular shape.

It is known from the requirements of clinical practice that when examining the thorax of a patient the FOV is normally set along the inner boundaries of the thorax in order to be able to obtain an optimum display area and a corresponding result. In the case of the technology currently to hand, the scan area and the FOV are set altogether in advance to a specific position. The corresponding size is also fixed in advance, as the rectangle 20 from FIG. 1 shows. However, the width and size of the thorax that is being scanned differ in each case.

Consequently, the operating staff of the CT unit must firstly fix the size of the scan area on the topogram of the relevant patient (that is to say the distance between the vertical sides of the rectangle 20), in order subsequently to set the size of the setting range for the FOV (that is to say the distance between the horizontal sides of the rectangle 20) in accordance with the actual conditions regarding width and size of the thorax. It is achieved thereby that the area of the FOV includes exactly all the inner boundaries of the thorax inside the scan area designated above.

The setting designated above for the FOV by manual regulation entails a high degree of deficiency. Moreover, in order to obtain an optimum display area and an appropriate result there is firstly a need to ensure that the boundaries of the FOV intersect the points of the outermost sides of the inner boundaries of the thorax of the patient. The boundaries of the manually set FOV are in no way adequately precise. Moreover, the manual setting of the boundaries of the FOV requires a high outlay on time, and this leads to a corresponding lengthening of the period required for the overall operation, and reduces the effectivity. Finally, the lengthening, designated above, of the overall operation leads to an additional physiological and psychological burden on the patient.

### SUMMARY

At least one embodiment of the present invention provides a method for automatically setting the FOV along the inner boundaries of the thorax on a CT topogram. As such, it may achieve, for example, a precise, highly efficient and quick setting of the FOV.

A method for automatically setting and reconstructing the field of view along the inner boundaries of the thorax, is proposed according to at least one embodiment of the invention, to carry out setting and reconstruction of the FOV inside a specific scan area of the CT topogram. The method includes:
a) searching for the point with the maximum CT value of each scan line inside the scan area and the CT value thereof on the topogram designated above;
b) searching for the point with the maximum CT value in the direction of the thorax, and finding the points whose CT value is the X-fold of the maximum CT value designated above, 0<X<0.5. The found points previously designated are located on the inner boundaries of the thorax;
c) repeating step a) to b) to search for points on the inner boundaries of the thorax inside the scan area designated above, these points forming the inner boundaries of the thorax; and
d) setting and reconstructing the field of view such that the field of view includes all found points on the inner boundaries of the thorax inside the relevant scan area.

A very good recognition effect is obtained here when the value for X is selected as 0.25 under step b). The points, described under step a), with the maximum CT value are located on the bones surrounding the inner boundaries of the thorax. When the setting and reconstruction of the field of view is carried out under step b), it is ensured that the boundaries of the reconstructed field of view intersect the points of the outermost side on the inner boundaries of the thorax inside the scan area. It is ensured in this way that the reconstructed field of view includes all the points on the inner boundaries of the thorax inside the relevant scan area.

When the maximum CT value described under step a) is greater than a predetermined metal CT value, it is assumed that metal is located in the relevant scan line, and in this case the boundary points of the previous scan line are considered as boundary points of the present scan line.

In a practical implementation example in accordance with at least one embodiment of the present invention, boundary points of the last scan line of an already scanned area are lengthened and form the boundary of an area not yet scanned. It is also possible that the data of a few lines of the start region and end region of the scan area of the topogram are erased in order to prevent deformation of linear artifacts.

In a further practical implementation example of at least one embodiment of the present invention, median filtering is undertaken in order in the course of the search operation to remove found discrete points that correspond to the relevant conditions.

After the inner boundaries of the thorax of the patient have been found in accordance with at least one embodiment of the present invention, it is possible to carry out the precise, highly efficient and quick setting of the FOV on the relevant topogram.

### BRIEF DESCRIPTION OF THE DRAWINGS

FIG. 1 is an overview of the manual setting of the FOV on a CT topogram in accordance with the present prior art.

FIG. 2 is an overview of the automatic setting of the FOV along the inner boundaries of the thorax on a CT topogram in accordance with at least one embodiment of the present invention.

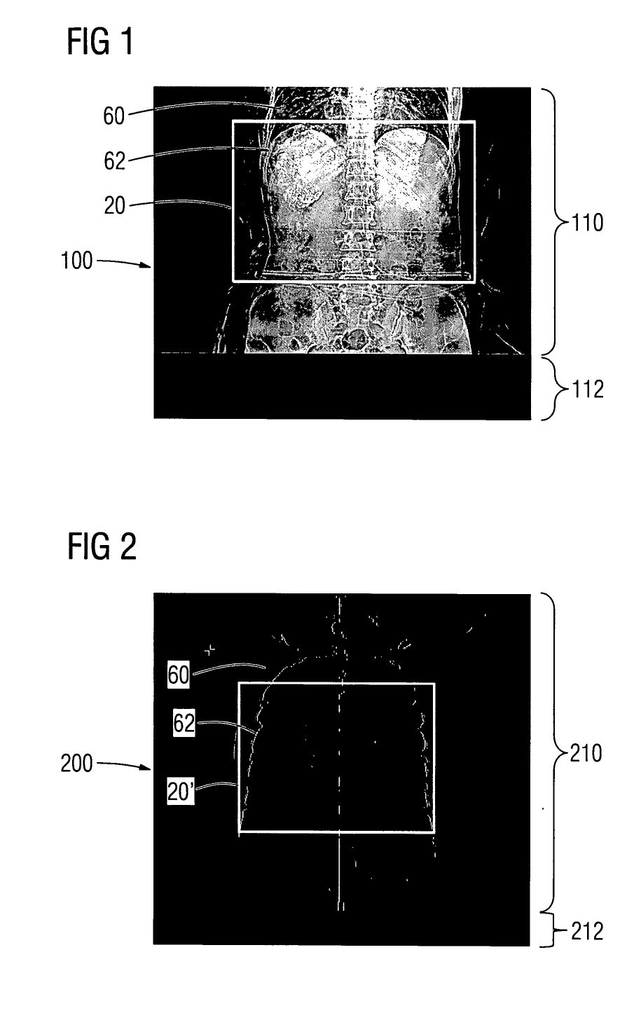

### DETAILED DESCRIPTION OF THE EXAMPLE EMBODIMENTS

The images of the topogram taken by CT units are displayed by way of various brightness levels. The various absorptivities of the body organs and of the body tissue with regard to x-rays are reproduced in this way. Dark shaded regions signify a low absorption on the topogram. These are regions of low density, for example lungs. Bright regions, by contrast, signify a high absorption. These are regions of high density, for example bones. In practice, it is normal to use CT values in order to display the intensity of the relevant density. CT values are normally given in a rising fashion on the topogram for air, fat, water, soft tissue and bone.

However, in at least one embodiment of the present invention, general findings for the field of body topography are used. For example, the inner boundaries of the thorax are surrounded by bones. Moreover, the CT values of bones are greater than the CT values of other regions and of sickbeds. The first step, in at least one embodiment of the present invention, is to set the relevant scan area on the topogram. Subsequently, the deviation specifications of the CT values of various regions of the patient's body are used for the purpose of determining the inner boundaries of the thorax of the relevant patient, and then to undertake the setting of the FOV to the inner boundaries of the thorax of the patient inside the scan area such that there is an overlap with the points on the outermost side of the left-hand and right-hand inner boundaries of the thorax inside the scan area. An improved or even optimum result can be achieved in this way with regard to scanning and an image reconstruction. For this reason, the main task of the automatic setting of the FOV resides in being able to undertake a precise localization of the left-hand and right-hand inner boundaries of the thorax of the patient on the topogram.

In at least one embodiment of the present invention, inner boundaries of the thorax refers to the inner contour lines of the thorax of the patient on the topogram. The inner boundaries of the thorax inside the scan area are the inner contour lines of the thorax of the patient inside the scan area set on the topogram previously designated.

In at least one embodiment of the present invention, the points of the inner boundaries of the thorax of the patient are determined along the body height of the patient. Here, the examination area is the region of the scan area set on the topogram designated above. The rate of linear artifacts in the regions where scanning starts and ends is normally higher than in the middle regions. The reason for this is that the inner boundaries of the thorax of the human body normally have no sudden changes and so the erasure of the data of a few scan lines from start and end regions does not entail any negative effects on the precision of the inner boundaries of the thorax in accordance with the present invention. Since, in addition, the body of the relevant patient is chiefly located in the middle part of the topogram, in the case of the present invention every scan line of the image is simultaneously subdivided into a right-hand and a left-hand part.

The points on the left-hand inner boundary of the thorax of the patient, and the points on the right-hand inner boundary of the thorax of the patient can respectively be found on the left-hand part, designated above, of a scan line and on the right-hand part of a scan line. Because the method for searching for the left-hand inner boundary of the thorax of the patient is identical to that for searching for the right-hand inner boundary of the thorax of the patient, only the description of the inventive method for finding the left-hand inner boundary of the thorax is set forth below as an exemplary design.

FIG. 2 serves for reference. In the relevant figure, number 210 signifies a scan region on the topogram 200. Number 212, by contrast, signifies the unscanned region on the topogram 200. Finding the left-hand inner boundary of the thorax of the patient's body 60 by way of the method according to at least one embodiment of the invention includes:
a) searching for the point with the maximum CT value of each scan line inside the scan area on the topogram 200 designated above, as well as the CT value thereof. Normally, the bones of the human body 60 supply the highest CT values.
b) Searching along the relevant points with maximum CT value from left to right—that is to say, from the bones, surrounding the inner boundaries of the thorax, of the patient's body 60 in the direction of the thorax on the topogram 200 designated above for points whose CT value is the X-fold of the maximum CT value designated above, in which case 0<X<0.5. The points found in this way are located on the inner boundaries 62 of the thorax of the patient's body 60. The CT values of various parts and tissues of the human body certainly differ, but there is a proportional relationship nevertheless. It is known from numerous experiments that points whose CT value is from 0 to 0.5 times the maximum CT value are located on the inner boundaries 62 of the thorax. An improved or even optimum recognition effect is achieved in practical application when the value of 0.25 is selected for X. The advantage that in the method according to the invention the search for the inner bone takes place at first, and the position of the inner boundaries 62 of the thorax is subsequently found, resides in that when searching inside the scan area of the topogram 200 from left to right or from right to left in accordance with the conditions of a correspondence of points with regard to the 0 to 0.5-fold of the maximum CT value it is prevented that points would firstly be found that are not located on the inner boundaries 62 of the thorax of the patient's body 60—such as for example, points of the sickbed outside the patient's body 60, which likewise fulfill the conditions with regard to the CT values designated above.
c) Repeating step a) to b) to search for points on the inner boundaries 62 of the thorax of the patient's body 60 inside the scan area designated above. These points form the inner boundaries 62 of the thorax of the patient's body 60.

The points for the right-hand inner boundary of the thorax of the patient's body 60 of each scan line of the topogram 200 can be found in a corresponding way with the aid of the same method. Consequently, the left-hand and right-hand inner boundaries of the thorax of the patient's body 60 are found and are used for the subsequent setting and reconstruction of the field of view.

Furthermore, the situation is such that when the maximum CT value designated above is greater in the step a) designated above than the predetermined metal CT value T_{Metal}, it is assumed that metallic material is located in the relevant scan line. This is so because the CT value of metal is higher than the CT value of human bones. Consequently, when metal is located in the relevant scan line and, furthermore, the corresponding metal CT value is used as a basis for calculating in accordance with step b), the points that are found which fulfill the condition of the 0 to 0.5-fold value of the relevant metal CT value are in no way necessarily points on the inner boundary 62 of the thorax of the patient's body 60.

Under these circumstances, the boundary points of the previous scan line are considered as boundary points of the relevant scan line. Because the boundaries of the thorax of the human body normally have no sudden changes, the approximate solution designated above proves to be sensible and acceptable.

Furthermore, the situation is such that when no scanning operation has taken place for some lines in the topogram 200 designated above, it is possible to lengthen the boundary point of the inner boundary 62 of the thorax of the last, already scanned line, and to consider it as inner boundary 62 of the thorax of the relevant scan line.

After the inner boundaries 62 of the thorax of the patient's body 60 have been obtained, the median filtering can optionally be performed in order to remove discrete points which have been found in the course of the search operation and correspond to the CT value conditions described under step a) or b), in order thereafter to set the FOV.

Finally, the FOV is set on the left-hand and right-hand inner boundaries of the thorax inside the scan area such that the FOV contains precisely all the points on the inner boundaries 62 of the thorax inside the scan area designated above. Thus, the FOV area intersects the points of the outermost side of the inner boundaries 62 of the thorax of the patient's body 60 inside the scan area. In order to achieve an optimum effect of image reconstruction, the procedure as in FIG. 2 number 20′ is adopted. In the course of the setting of the FOV designated above, the positioning is set in a dynamic way while regulating the scan area, and while this is being done the FOV area intersects the points of the outermost side of the inner boundaries 62 of the thorax of the patient's body 60 inside the scan area.

After the setting of the scan area on the topogram 200 designated above and after setting of the FOV 20′, the serial or spiral scanning can subsequently be carried out.

Of course, the inner boundaries 62 of the thorax determined by way of the method according to at least one embodiment of the invention need not necessarily run in a continuous fashion. FIG. 2 may be regarded in this connection. The inner boundaries 62 of the thorax certainly do not run continuously, but are bent.

Moreover, some of the found points are located in the middle of the topogram inside the scan area. The reason for this is that in the case of the relevant scan lines the CT values are greatest for the spinal column of the patient's body 60, and the points whose CT value is the 0 to 0.5-fold of the maximum CT value are located in the vicinity of the spinal column.

However, this circumstance in no way impairs the setting of the FOV in accordance with the method of at least one embodiment of the present invention, because the points for setting the FOV of the method according to at least one embodiment of the invention are located on the outermost sides of the inner boundaries 62 of the thorax. Consequently, there is no occurrence of impairment of the setting of the FOV in accordance with the method according to at least one embodiment of the invention even should points that correspond to the conditions be found outside the inner boundaries 62 of the thorax.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDS; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for automatically setting and reconstructing the field of view along the inner boundaries of the thorax on a CF topogram for setting and reconstructing the field of view inside a specific scan range on the topogram along the inner boundaries of the thorax, the method comprising:
a) searching for a point with a maximum CT value of each scan line inside the scan area and a CT value thereof on the topogram;
b) searching for a point with a maximum CT value in a direction of the thorax, and finding points whose CT value is an X-fold of a maximum CT value designated above, 0<X<0.5 and the found points designated above are located on the inner boundaries of the thorax;
c) repeating step a) to b) to search for points on inner boundaries of the thorax inside the scan area designated above; and
d) setting and reconstructing the field of view such that the field of view includes all found points on the inner boundaries of the thorax inside the relevant scan area.

2. The method for automatically setting and reconstructing the field of view along the inner boundaries of the thorax on a CT topogram as claimed in claim 1, wherein the points, described under step a), with the maximum CT value are located on the bones surrounding the inner boundaries of the thorax.

3. The method for automatically setting and reconstructing the field of view along the inner boundaries of the thorax on a CT topogram as claimed in claim 1, wherein, when the maximum CT value described under step a) is greater than a previously determined metal CT value, it is assumed that metal material is located in the relevant scan line, and wherein, in this case, the boundary points of the previous scan line are considered as boundary points of the present scan line.

4. A method for automatically setting and reconstructing the field of view along the inner boundaries of the thorax on a CT topogram as claimed in claim 1, wherein the data of a few lines of the start region and end region of the scan area of the topogram are erased in order to prevent deformation of linear artifacts.

5. The method for automatically setting and reconstructing the field of view along the inner boundaries of the thorax on a CT topogram as claimed in claim 1, wherein median filtering is further undertaken in order in the course of the search operation to remove found discrete points that correspond to the CT value conditions described under step b).

6. A method for automatically setting and reconstructing the field of view along the inner boundaries of the thorax on a CT topogram as claimed in claim 1, wherein, when setting and reconstructing the field of view in point d), the boundaries of the reconstructed field of view intersect the points of the outermost side on the inner boundaries of the thorax inside the scan area.

7. The method for automatically setting and reconstructing the field of view along the inner boundaries of the thorax on a CT tomogram as claimed in claim 1, wherein X, under step b), is selected as 0.25.

8. The method for automatically setting and reconstructing the field of view along the inner boundaries of the thorax on a CT topogram as claimed in claim 6, wherein X, under step b), is selected as 0.25.

9. A method for automatically setting and reconstructing the field of view along the inner boundaries of the thorax on a CT topogram as claimed in claim 2, wherein, when setting and reconstructing the field of view in point d), the boundaries of the reconstructed field of view intersect the points of the outermost side on the inner boundaries of the thorax inside the scan area.

10. A method for automatically setting and reconstructing the field of view along the inner boundaries of the thorax on a CT topogram as claimed in claim 3, wherein, when setting and reconstructing the field of view in point d), the boundaries of the reconstructed field of view intersect the points of the outermost side on the inner boundaries of the thorax inside the scan area.

11. A method for automatically setting and reconstructing the field of view along the inner boundaries of the thorax on a CT topogram as claimed in claim 4, wherein, when setting and reconstructing the field of view in point d), the boundaries of the reconstructed field of view intersect the points of the outermost side on the inner boundaries of the thorax inside the scan area.

12. A method for automatically setting and reconstructing the field of view along the inner boundaries of the thorax on a CT topogram as claimed in claim 5, wherein, when setting and reconstructing the field of view in point d), the boundaries of the reconstructed field of view intersect the points of the outermost side on the inner boundaries of the thorax inside the scan area.

13. The method for automatically setting and reconstructing the field of view along the inner boundaries of the thorax on a CT tomogram as claimed in claim 2, wherein X, under step b), is selected as 0.25.

14. The method for automatically setting and reconstructing the field of view along the inner boundaries of the thorax on a CT tomogram as claimed in claim 3, wherein X, under step b), is selected as 0.25.

15. The method for automatically setting and reconstructing the field of view along the inner boundaries of the thorax on a CT tomogram as claimed in claim 4, wherein X, under step b), is selected as 0.25.

16. The method for automatically setting and reconstructing the field of view along the inner boundaries of the thorax on a CT tomogram as claimed in claim 5, wherein X, under step b), is selected as 0.25.

17. A computer program to, when executed on a computer, cause the computer to carry out the method as claimed in claim 1.

18. A computer program product, including the computer program of claim 17.

19. A computer readable medium including program segments for, when executed on a computer, causing the computer to implement the method of claim 1.
